(19) United States
(12) Patent Application Publication
(10) Pub. No.: US 2004/0101542 A1
(43) Pub. Date: May 27, 2004

(54) Skin care composition with retinyl ester as vitamin a propionate retinyl palmitate d-alpha tocopherol rice amino acids and liposomes and method of application

(76) Inventors: Beryl Ann Breusch, Naples, FL (US); Cynthia A Rager, Ft Myers, FL (US)

Correspondence Address: Kevin S Lemack, Nields & Lemack, Suite 7, 176 E Main Street, Westboro, MA, 01581, US

(21) Appl. No.: 10/466,727

(22) PCT Filed: Feb. 14, 2002
(86) PCT No.: PCT/US02/04346

(51) Int. Cl. A61K 31/355; A61K 31/23

(52) U.S. Cl. 424/401; 514/458; 514/552

(57) ABSTRACT

A skin conditioning composition combining specific percentages of a retinyl ester as vitamin A propionate, retinyl palmitate, vitamin E as d-alpha tocopherol, with rice amino acids and liposomes to create an efficient vehicle for skin rejuvenation without common complications of typical retinoid therapy. Also discloses is a method of application.

### BACKGROUND OF THE INVENTION

It is well known that retinol and retinyl esters enhance keratinocyte proliferation and the synergistic inhibition of keratinocyte differentiation. Retinoic acid has been shown to be most effective in producing these results which reduce the occurrance of acne. However, substantial irritation and erythema can occur at sufficient strengths to produce therapeutic results. Recent use of retinoids has suggested treatment for photoaging and sun damage. Examples of this are characteristic wrinkling, loss of elasticity, roughened texture and hyperpigmentation. U.S. Pat. No. 6,080,393 to Liu, et al. addresses the preference of certain retinyl esters, retinol and retinal as preferred forms in formulating skin care compositions with these naturally occurring retinoids.

Delivering stable vitamin A in therapeutic strengths for reduction of photoaging and improvement of skin texture without side effects has been problematic. Fulton, Jr. addresses this in U.S. Pat. No. 5,043,356. He states that vitamin A propionate has clinically demonstrated unexpectedly superior properties for the treatment of skin disorders with markedly reduced side effects. Dermal alterations resulting from excessive sun exposure can be corrected with an accompanying improvement in the skin appearance. The molecular weight of vitamin A propionate, which is an ester of retinol and propionic acid, is the correct molecular weight and configuration to remain in a stable solution and to be transdermally delivered while it is active.

Increasing strengths of vitamin A propionate can increase the therapeutic benefits of retinoic treatment, but in increasing proportions, the typical side effects of retinoid therapy still occur, such as increased surface tension, irritation, dry, scaling and erythema.

When increasing retinyl ester strength for more therapeutic value, it has been found that surface irritation can be decreased by ameliorating the less desirable side effects with larger molecule retinyl palmitate and d-alpha tocopherol in combinations so that the ratio of the palmitate/tocopherol mixture remains constant.

### SUMMARY OF THE INVENTION

The problems of the prior art have been overcome by the present invention, which provides a cosmetic preparation shown to significantly enhance skin quality without substantial irritation, and a method of applying the preparation to the skin. The compositions of the present invention include a therapeutically effective amount of a retinyl ester as vitamin A propionate that minimizes or eliminates the side effects commonly associated with vitamin A. An optimal formula for addressing photoaging of skin contains percentages of a retinyl ester as vitamin A propionate at 4.5%-6.0%.

For amelioration of possible side effects, several additional components can be used in the invention. Because retinoid compounds can be unstable, an oil-in-water emulsion is the preferred formula. When using an oil-in-water emulsion along with an antioxidant, the retinoid composition remains fairly stable. The larger molecule of retinyl palmitate and d-alpha tocopherol tend to remain at surface levels for softening side effects of irritation and erythema. Additionally, they provide antioxidant benefit and increase surface humidity, thereby aiding in delivery through the stratum corneum. Because the stratum corneum is the first rate-limiting membrane of the epidermis, the condition of the skin affects the actual percutanious absorption. Hydrated skin increases percutaneous absorption potential (E M Jackson, Percutaneous absorption: scientific basis for new products or concern for safety, presented at the 38^{th} International CIDESCO Conference, New York, N.Y. (Aug. 20, 1984). The amount of retinyl palmitate and d-alpha tocopherol found to be most beneficial is a constant ratio of 1:1:1 to that of vitamin A propionate.

In its method aspects, the present invention relates to the topical application to the skin of a patient a formulation containing a therapeutically effective amount of vitamin A proprionate, together with vitamin A palmitate and d-alpha tocopherol, most preferably in about a 1:1:1 weight ratio.

### DETAILED DESCRIPTION OF THE INVENTION

A predominant emulsion type is the oil-in-water emulsion, where the dispersed phase is oil and the surrounding phase is water. The oil and water are held together by applying physical energy and using chemical emulsifiers, which have hydrophilic and lipophilic ends to bind both the oil and water (Zatz, Joel L., Skin Permeation Fundamentals and Appication, Wheaton, Ill., Allured Publishing, 1993). Percutaneous absorption is governed by the principles of passive diffusion. The stratum corneum controls percutaneous absorption to a greater degree than the other rate-limiting barriers of the skin. The reciprocal function of the stratum corneum is also its reservoir function. This function allows concentrations of the retinyl ester to remain in the stratum corneum as concentration of the permeant decreases with each successive epidermal stratum.

Liposomes act as a delivery agent in the preparation. Liposomal formulations, when topically applied, exhibit unique properties beneficial to a wide variety of cosmetic applications. They encapsulate high concentrations of water and lipid soluble substances and facilitate their delivery into the skin. Liposomes are microscopic vesicles composed of one or more lipid bilayers arranged in concentric fashion enclosing an equal number of aqueous compartments. (A D Bangham, M M Standish and J C Watkins, the action of steroids and steptolysin S on the permeability of phospholipid structures to cations, J Mol Biol 13 138 (1965)). When mixed at high speeds, they encapsulate the vitamin A palmitate and d-alpha tocopherol. Vegetable phospholipids are widely used for topical applications in cosmetics and dermatology since they have a high content of esterfied essential fatty acids, especially linoleic acid. (M M Rieger, Skin lipids and their importance to cosmetic science, Cosm & Toil 102 36 (1987); H Lautenschlager, J. Roding and M. Ghyczy, The use of liposomes from soya phospholipids in cosmetics, SOFW 14 531 (1988)). Liposomal deposition into the stratum corneum results in a substantial reservoir effect. A skin lipid formula containing ceramides, cholesterol, palmitic acid and cholesteryl sulfate in the weight ratio of 4:2.5:2.5:1 can be used by preparing with a conventional film method. In a study reported by Egbaria, Weiner, the uptake and distribution of retinoic acid in the deeper skin strata at 24 hours was generally 3-4 times higher from liposomal formulations than from solutions or emulsions (K. Egbaria, C. Ramachandran and N. Weiner, Topical application of liposomally entrapped ciclosporin evaluated by in-vitro diffusion studies with human skin, Skin Pharmacol 4 21 (1991)).

Rice protein as rice amino acids have been shown to aid in skin firmness, moisturization and to increase microcirculation. A product such as by trade name, Aqua Pro II by Midwest Grain, at 1.0% to 5.0% in formulation can decrease substantial irritation while aiding in the primary objective of firmness.

Suitable thickening agents can be chosen from cellulosic derivatives such as hydroxyethyl cellulose, methyl cellulose, carboxymethylcellulose and sodium carboxlymethylhydroxyethylcellulose. They may also be chosen from natural gums or starches such as guar, carrageenum or rice starch.

Additional moisturizing agents and solvents may be utilized for preparation of phases, such as propylene glycol. Other oils may be utilized, as an example, rice bran oil, which contains ferulic acid and phytic acid known as food preservative, antioxidant, stabilizing and chelating.

The compositions of the present invention can be prepared by mixing or blending procedures known to those skilled in the art. Preferably the aqueous phase is prepared by weighing the appropriate amount of water and mixing in the cellulose until uniform. The mixture is heated to about 70-75° C., and is then cooled to about 40° C. Preferably the oil phase is prepared by mixing the appropriate amounts of oil (e.g., castor oil), vitamin A propionate, retinyl palmitate and d-alpha tocopherolin a suitable vessel at a temperature of about 65° C. This mixture is then cooled to about 35° C. Methyl and propylene glycol, for example, are added to the aqueous mixture, mixed, and the oil mixture is then added under agitation. Liposomes, such as Bio Botanicals, are then added and mixed at high speed, and the resulting formulation is allowed to settle.

Frequency of use can depend upon factors including the skin type and skin condition of the patient, patient age, and the climate. The following application procedure is preferred, with exceptions for age, climate and skin type noted accordingly:

Morning Procedure

For the morning procedure usage of the composition of the present invention is the same for all skin types, ages, and climates. Patients with sensitive skin or rosacea should use the inventive composition every other morning for the first 2 to 3 weeks. After 2 to 3 weeks, they should start using the composition every morning, as the skin's tolerance of the composition will increase over time.

There are four simple steps in the morning:

Step 1: Before cleansing, apply Enzyme, especially under the eyes and all over the face. The Enzyme has the following composition:

| ITEMS | SHARED (By Weight) |
|---|---|
| Distilled Water | 39% |
| Cranberry-Resveratrol Complex | 40% |
| 1-Glucoronic | 11.5% |
| 1-Lactic Acid | 3.8% |
| Rye Polyglucose | 4.2% |
| Jasmine Oil | 1.5% |

Care must be taken not to get any Enzyme in the eye. The Enzyme is left on for approximately 3 to 5 minutes. If sensitivity or irritation occurs, reduce the time the Enzyme is left on the skin. Immediately remove the Enzyme by showering or rinsing the skin. The Enzyme helps remove dead skin.

Step 2: Follow with the Cleanser using a washcloth with warm water. The Cleanser has the following composition:

| ITEMS | SHARED (By Weight) |
|---|---|
| Demineralized Water | 43% |
| TEA Lauryl Sulfate | 15% |
| Cocamidopropyl Betaine | 13% |
| Oleamide D.E.A. | 4.07% |
| Aloe Vera | 7.5% |
| Glycerine | 4.85% |
| Sage Extract | 1.2% |
| Cucumber Extract | .6% |
| Lactic Acid | 4.5% |
| Green Tea Extract | 1.5% |
| Tea Tree Oil | 2% |
| Citric Acid | .04% |
| Salicylic Acid | .9% |
| Polysorbate 20 | .03% |
| Sodium Ascorbate | .06% |
| Vitamin E Acetate | .05% |
| Glycerin | .22% |
| Methylchlorisothazolinone | .55% |
| Methylisothiazolinone | .08% |
| Methylparaben | .03% |
| Propylparaben | .8% |
| Fragrance | .02% |

After cleansing, use warm water with a washcloth and soak your skin for 2 to 3 minutes. Make sure to soak (more than rub) the skin. Skin irritation may occur if the skin is rubbed too aggressively.

Step 3: Using the forefinger, apply 1 pump of the skin care composition of the invention under each eye by laying it close to the lower eyelashes. Let the composition absorb into the eye area. Do not rub. Rubbing will spread the product away from your eye area. Continue by applying 4 to 6 pumps of the composition all over the face. Do not rub. Wait 5 minutes for the composition to absorb.

Step 4: After the composition has absorbed apply Therapy Gel as a moisturizer and sunscreen over the composition. The Therapy Gel has the following composition:

| ITEMS | SHARED (By Weight) |
|---|---|
| Deionized Water | 32% |
| Aloe Vera | 22% |
| Propylene Glycol | 12% |
| Shea Butter | 8.5% |
| Sunflower Fatty Acids | 5% |
| Allantoin | 2.3% |
| Beeswax | 1.5% |
| Ceresin | 1.5% |
| d-alpha tocopherol | 2.85% |
| Cellulose | 2% |
| Cucumber Oil | 1% |
| Calendula Extract | .9% |
| Citroen | .45% |
| Zinc Oxide | 3% |
| Titanium Dioxide | 5% |

Night Procedure

For the night procedure usage of the inventive composition varies according to skin types, ages and climates and consists of 2 programs. A special note: all patients must begin with Program 1 for the first 2 weeks to gradually build the skin's tolerance to the product.

Program 1: Recommended for Colder, Dryer Climates, Ages Under 30, Sensitive Skin or Rosacea.

This program does not use the skin care composition of the invention at night. Cleanse with the Cleanser using a washcloth. After cleansing, soak your skin with warm water for 3 to 4 minutes. Apply Therapy Gel every night and leave on. Do not apply the Enzyme at night.

After 2 weeks if you are over 30, have normal to oily skin and do not live in a colder dryer climate you may proceed to Program 2.

Program 2: Recommended for Ages Over 30, Normal to Oily Skin and Most Climates.

This program uses the product every other night in addition to in the morning. Cleanse with the Cleanser using a washcloth. After cleansing, soak your skin with warm water for 2 to 3 minutes. The first night, apply the product (see Step 3 under Morning Procedure). Follow with Therapy Gel over the product. The second night apply only Therapy Gel and leave on. Continue to alternate first and second night procedures (using the product every other night.) Do not apply the enzyme at night.

The inventive composition may be applied 2 times per week on the neck area.

### EXAMPLE 1

In patients clinically observed for use of product the following has been reported:

In a total of 40 patients who have used a topically applied formula with about 6.0% vitamin A propionate, retinyl palmitate and d-alpha tocopherol

10 patients have been able to use the formula one time per day topically applied with no significant irritation

23 patients have been able to use the formula two to four times per week with no significant irritation

6 patients have been able to use the formula one to two times per week with no significant irritation

1 patient used the formula one time per week or less with no significant irritation

All patients reported some visible change after use of product for a period of six weeks or longer. Self-reporting claims are smoother, tighter skin and less visible lines.

### EXAMPLE 2

A focus group utilizing a formulation of the invention in a serum of the following composition was executed:

| | |
|---|---|
| Distilled water: | 56% |
| Castor Oil: | 14% |
| Methyl glycol: | 9% |
| Vitamin A propionate: | 6% |
| Vitamin A palmitate: | 6% |
| d-alpha tocopherol: | 6% |
| Cellulose: | 5% |
| Rice amino acids: | 4% |
| Liposomes: | 1% |

The purpose of the group trial was to substantiate and validate cosmetic claims concerning benefits and usage of the product. It was a self-evaluating and self-reporting group.

All participants followed the defined instructions of application which include frequency of use and which are integral to the results of the product claims:

All participants agreed to compliancy and used only the following products on their skin:

Serum—a retinyl ester serum with the following composition:

| ITEMS | SHARED (By Weight) |
|---|---|
| Distilled Water | 59% |
| Castor Oil | 14% |
| Methyl Glycol | 9% |
| Vitamin A Propionate | 6% |
| Vitamin A Palmitale / d-alpha tocopherol | 6% |
| Liposomes | 1% |
| Cellulose | 5% |

Cleanser—a mild cleanser of gel consistency, suitable for any skin type, with the composition shown above

Therapy Gel—a gel cream consistency moisture product, with the composition as shown above

The Cleanser and the Therapy Gel product were given as a control to replace the cleansing and moisture products that the participant normally would have been using.

All participants had the same application method and instructions and used the products for a period of six weeks.

A.M. Instructions for Every Day of Trial:

1. Use warm water and wash cloth to soak skin, apply approximately 2 ml. of cleanser to face and cleanse. Rinse again with warm water. Apply warm water in compress form with cloth for about 2 minutes before drying with towel.

2. Apply a total of 5 metered pumps of serum to entire face and under eye area (0.3 ml metered pump dose). Allow to absorb. Do not apply to eye lids.

3. Apply moisture gel in light application over entire area.

P.M. Instructions for Weeks 1 and 2:

1. Follow step 1 of A.M. instructions for cleansing.

2. Follow step 3 of A.M. instructions for moisture.

P.M. Instructions for Weeks 3, 4 and 5:

1. Follow step 1 of A.M. instructions for cleansing.

2. Follow step 2 of A.M. instructions for serum application. Apply every other night only.

3. Follow step 3 of A.M. instructions for moisture.

A technician evaluation prior to start of program and at the end of the six-week trial, along with before and after pictures, were used as a base line reference.

Of 15 people who participated in the study, 7 fell in the defined parameters for age category, which was 35 -54. These participants of Study One had a variety of normal to dry skin types. Their FitzPatrick scale values ranged from I-III, with light to moderate hyperpigmentation. Of these participants, 6 out of 7 (85.5%) reported some improvement in one week. All participants (100%) reported moderate to strong improvement in at least 4 of the defined categories at the end of 6 weeks. No participants reported dry, irritable skin with any erythema, conditions typically associated with retinoid therapy. One participant reported an acne break out at 3 weeks. No erythema or irritation was noted by technician.

The results of the self-reported categories for Study One are in Table A and Table B.

TABLE A
Study One

| Condition | S | % | M | % | N | % |
|---|---|---|---|---|---|---|
| Fine Lines | 1 | 14% | 6 | 86% | 0 | 0% |
| Wrinkles | 1 | 14% | 4 | 57% | 2 | 29% |
| Smoothness | 1 | 14% | 6 | 86% | 0 | 0% |
| Firmness | 1 | 14% | 5 | 71% | 1 | 14% |
| Texture | 1 | 14% | 6 | 86% | 0 | 0% |
| Discoloration | 1 | 14% | 2 | 28% | 4 | 57% |

Key: S (strong improvement)
M (moderate improvement)
N (no improvement)

TABLE B
Study One

| | Participants Reporting |
|---|---|
| Condition | Moderate-Strong Improvement in Six Weeks |
| Fine Lines | 100% |
| Wrinkles | 71% |
| Smoothness | 100% |
| Firmness | 85% |
| Texture | 100% |
| Discoloration | 42% |

The study was repeated with an additional 8 participants who did not fall within the defined age parameters. These participants had a variety of normal, oily and dry skin types. Their FitzPatrick scale varied from I-III. They had light to moderate hyperpigmentation noted.

Of this group, Study Two, 7 out of 8 (87.5%) reported some improvement in the first week. Six out of 8 (75%) reported strong to moderate improvement in at least 4 of the defined categories at the end of 6 weeks. One participant reported light itching as a complication. One participant reported an acne break out at one week. No participants reported dry skin or erythema, conditions typically associated with retinoid therapy. No erythema or irritation was noted by the technician.

The results of the self reported group Study Two are in Table C and Table D.

TABLE C
Study Two

| Condition | S | % | M | % | N | % |
|---|---|---|---|---|---|---|
| Fine Lines | 4 | 40% | 3 | 37.5% | 1 | 12.5% |
| Wrinkles | 3 | 37.5% | 2 | 25% | 3 | 37.5% |
| Smoothness | 5 | 62.5% | 3 | 37.5% | 0 | 0% |
| Firmness | 5 | 62.5% | 1 | 12.5% | 2 | 25% |
| Texture | 4 | 50% | 2 | 25% | 2 | 25% |
| Discoloration | 1 | 12.5% | 2 | 25% | 5 | 62.5% |

Key: S (strong improvement)
M (moderate improvement)
N (no improvement)

TABLE D
Study Two

| | Participants Reporting |
|---|---|
| Condition | Moderate to Strong Improvement in Six Weeks |
| Fine Lines | 87.5% |
| Wrinkles | 62.5% |
| Smoothness | 100% |
| Firmness | 75% |
| Texture | 75% |
| Discoloration | 37.5% |

In conclusion, 86.5% of all participants reported some improvement in the first week of use. At the end of six weeks, in an average of both studies, participants reported a moderate to strong improvement in:

| | |
|---|---|
| Fine Lines | 93.5% |
| Wrinkles | 66.8% |
| Skin Smoothness | 100% |
| Firmness | 80% |
| Texture | 87.5% |
| Hyperpigmentation | 52.3% |

Reported complications were 13% reporting an increase in acne break out in weeks 1 -3, which resolved by week 6. Also reported was one participant (6%) with itching, which resolved by week 6 with no additional treatment.

It is significant to report that the category with the least change was improvement in discoloration (hyperpigmentation), although a change of over 52% improvement was noted. It is also significant that 100% of participants reported an improvement in skin smoothness. Smoothness was defined by participants as a tactile improvement in overall softness and moisture with no apparent areas of dry skin. It is also significant to report a total improvement of 93.5% in fine wrinkles, which was defined by participants as lines around the eye area.

What is claimed is:

1. A topically applied composition for treating photoaging and skin regeneration without substantial irritation of a cosmetically acceptable vehicle in an oil in water emulsion comprising:
from about 4.1% to about 8 by weight retinyl palmitate;
from about 4.1% to about 8.0% by weight d-alpha tocopherol; and
from about 4.1% to about 8.0% by weight vitamin A propionate.

2. The composition of claim 1 wherein said product has a pH of about 4.8 [symbol] 6.

3. The composition of claim 1 wherein the proportion of retinyl palmitate to d-alpha tocopherol remains equal.

4. The composition of claim 1, further comprising a thickener selected from the group consisting of hydroxyethyl cellulose, natural gums, guar, carrageenum and rice starch.

5. The composition of claim 1, further comprising acceptabl a moisturizing agent.

6. The composition of claim 1, further comprising oil.

7. The composition of claim 1, further comprising propylene gylcol.

8. The composition of claim 1, wherein the ratio of retinyl palmitate, d-alpha tocopherol and vitamin A proprionate is about 1:1:1.

9. The composition of claim 1, further comprising:
from about 0.1% to 5.0% by weight rice amino acids;
from about 0.1% -5.0% acceptable thickner;
from about 0.5% to 1.0% liposomes; and
from about 50.0% to 90.0% water.

10. A method of condition the skin of a patient, comprising:
providing a composition comprising:
from about 4.1% to about 6.0% by weight retinyl palmitate;
from about 4.1% to about 6.0% by weight d-alpha tocopherol; and
from about 4.1% to about 6.0% by weight vitamin A propionate; and
topcially applying said composition to the skin of said patient.

11. The method of claim 10, wherein said composition further comprises:
from about 0.1% to 5.0% by weight rice amino acids;
from about 0.1% -5.0% acceptable thickner;
from about 0.5% to 1.0% liposomes; and
from about 50.0% to 90.0% water.

12. The method of claim 10, wherein said retinyl palmitate, d-alpha tocopherol and vitamin A propionate are present in a weight ratio of about 1:1:1.
